(19) United States
(12) Patent Application Publication
(10) Pub. No.: US 2007/0270636 A1
(43) Pub. Date: Nov. 22, 2007

(54) Endoscopic Instrument

(76) Inventor: Gianluca Spera, Milan (IT)

Correspondence Address: Leffert Jay & Polglaze, P O Box 581009, Minneapolis, MN, 55458-1009, US

(21) Appl. No.: 10/551,893

(22) PCT Filed: Apr. 4, 2003
(86) PCT No.: PCT/IT03/00206

§ 371 Date: Jul. 25, 2007

(51) Int. Cl. A61B 1/005

(52) U.S. Cl. 600/104

(57) ABSTRACT

An endoscopic instrument (10), particularly useful for the non-invasive treatment of some digestive system pathologies, has a flexible and elongate main body (11) that houses a vision device (22) for taking images of an organ internal area, comprising a first working arm (21) for the use of tools, and further comprises a second working arm (23) for the use of tools apt to be operated independently with respect to said first working arm (21), allowing an endoscopic treatment of a wide range of pathologies that at present are treatable only surgically.

The present invention refers to an endoscopic instrument, in particular to an advanced endoscope particularly useful for the non-invasive treatment of some digestive system pathologies.

To date, nearly every endoscopic instrument is made according to a common and time-honored architecture.

Image management has evolved, also due to the normal taking over of new technologies, however instrument mechanics and architecture remained the same.

Front vision endoscopes (e.g. the common gastroscope or the colonscope), and side vision instruments (the duodenoscope) are known.

Besides from the above, miniaturized endoscopes (e.g. cholangioscope) in all reproducing the latter were made.

Common denominator of all known instruments for digestive endoscopy is the presence of a single working channel (it also having a front or side exit) always having a direction consensual to the instrument body. In fact, such channel is internal to the endoscope body and passively submitting to its motions. The working channel allows the use of tools and/or of other accessories like pincers, balloons, extraction baskets or lancets for carrying out therapeutic maneuvers.

To date, the therapeutic scope of digestive endoscopy covers treatments like, e.g. the draining of biliary lithiasis, the treatment of chronic pancreatitis, the palliation of biliary tract neoplastic stenoses by positioning prostheses, the resection of incipient neoplastic transformation areas (e.g. mucosectomy) or of adenomatous areas (e.g. ampullectomy, polipectomy).

To date, also the treatment of bleeding, the dilation of benign stenoses and the reduction of gastroesophageal reflux are carried out by endoscopy.

However, the great limitation of operative endoscopy remains that of not allowing the resection of wider areas of organs, as a single working channel having reduced mobility would not allow managing potential complications.

For these and other situations laparoscopic or laparotomic surgery has to be resorted to.

This however increases costs, lengths of hospital stay, and the general discomfort of the patient, who is unable to choose effective non-invasive and conservative techniques.

Object of the present invention is to solve the described known-art problems, providing an endoscopic instrument having a flexible and elongate main body that houses a vision device for taking images of an organ internal area, comprising a first working arm for the use of tools, characterized in that said main body comprises a second working arm for the use of tools apt to be operated independently with respect to said first working arm.

The instrument according to the present invention is an operative endoscopic device having advanced functions and an application scope lying between the endoscopic and the laparoscopic surgery fields.

The main advantage of an instrument according to the present invention, with respect to the known-art endoscopic instruments, is provided by the fact that it allows to endoscopically treat a range of pathologies that at present are treatable only surgically, thereby giving surgical/therapeutic ends to a specialty originally meant to have substantially diagnostic aims.

Hence, an instrument according to the present invention allows to transfer the features and the advantages of some surgical techniques, especially of modern laparoscopy, to the field of endoscopy

This is made possible mainly by virtue of the employ of two independent working arms that allow laparoscopy-like maneuvers inside of endoscopically investigated organs.

Further advantages, features, and the operation modes of the present invention will be made apparent in the following detailed description of preferred embodiments thereof, given by way of example and not for limitative purposes, making reference to the figures of the annexed drawings, wherein:

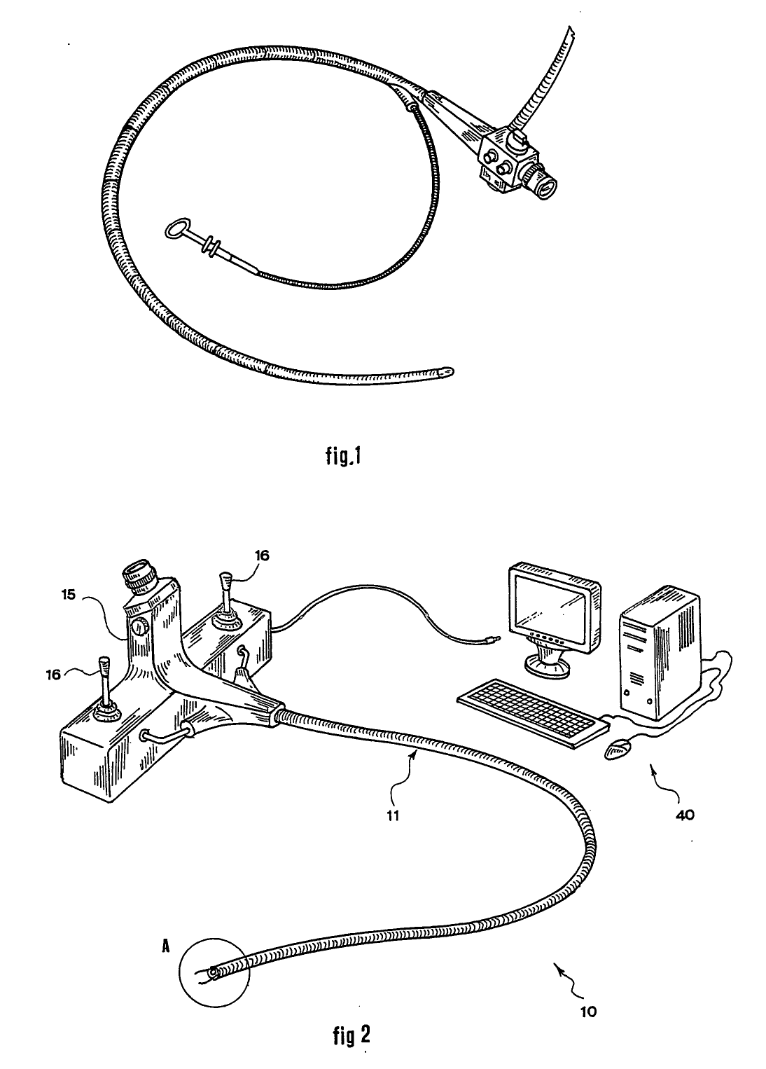

FIG. 1 shows a known-art endoscope;

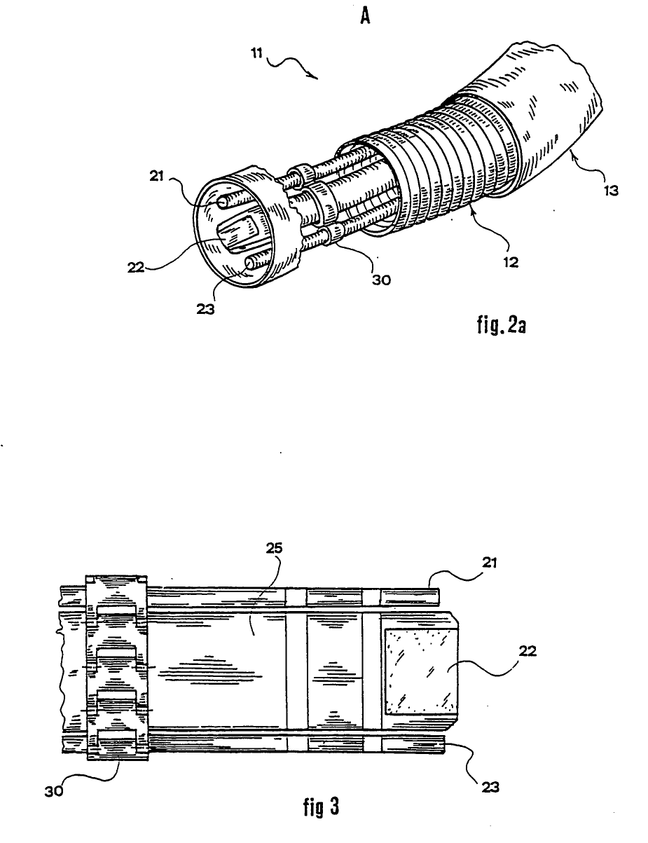

FIG. 2 is an exemplary sketch of a system for actuating and controlling an instrument according to the present invention;

FIG. 2A is a cross section of the working end portion of an endoscopic instrument according to the present invention;

FIG. 3 is a detail of the working end portion of an instrument according to the present invention in a resting position;

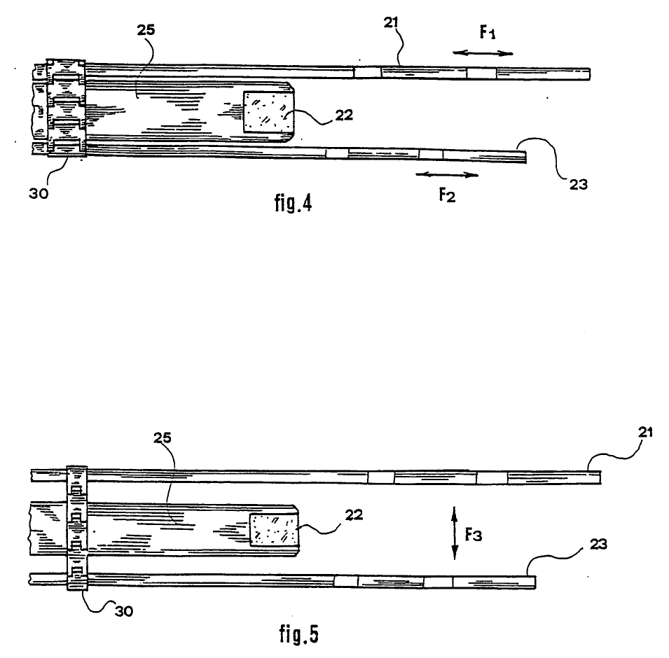

FIG. 4 is a detail of the working end portion with the two working arms extended;

FIG. 5 is a detail of the working end portion with the two working arms spaced from the central body;

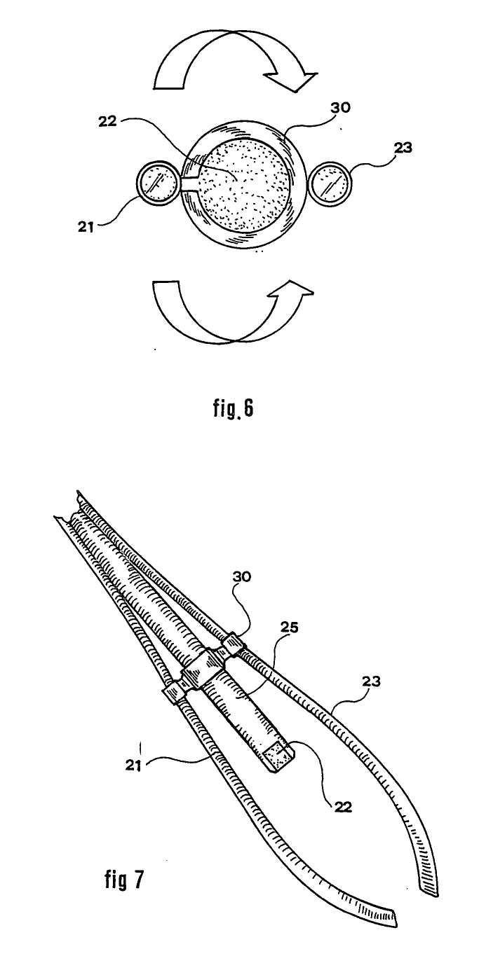

FIG. 6 is a sketch of an annular mechanism for connecting the working arms;

FIG. 7 is a detail of the working end portion with the two working arms in a working position;

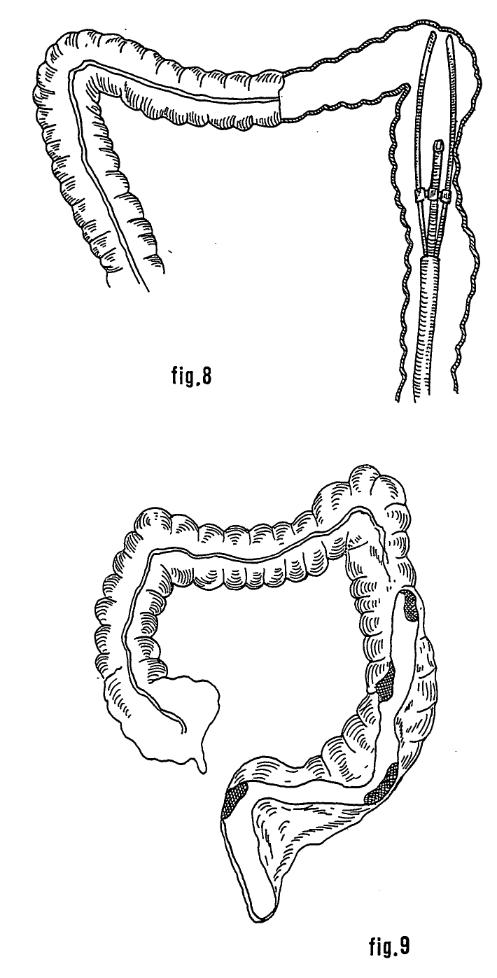

FIG. 8 is an exemplary view of the use of an instrument according to the present invention; and

FIG. 9 is an optional virtual visualization of the path of the instrument in the explored organ.

With initial reference to FIG. 1, it shows a known-art endoscope.

Hence, an endoscope 1 is essentially made of a flexible main body, comprising a working arm and a vision device, and of a manual apparatus 2 for motion control and image viewing.

With reference to the subsequent figures, an endoscopic instrument 10 according to the present invention comprises a flexible and elongate main body 11, having an annular metal skeleton 12 coated with an external sheath 13 preferably obtained from a rubber mix, such as to easily perform spatial motions.

For simplicity's sake, hereinafter reference will be made to an endoscope, this term being meant to refer to an endoscopic instrument according to the present invention.

The handling of the main body 11 is attained by a control system comprising first handling means (not shown in the figures), e.g. a system having one or more tie rods, mechanically operated by a user via controls 15.

Such a tie rod system will not be detailed, since its embodiment is substantially equivalent to those of known-art endoscopes and therefore within the capacity of a person skilled in the art.

Alternatively, or even in association to the tie rod system, such first handling means may advantageously comprise first motion actuating and controlling devices of electronic and/or electromechanical type.

In that case, the motions of the main body will be servoassisted, and the user will control such motions via electronic controls, e.g. a joystick or the like.

Such devices and the corresponding actuating and controlling instruments are directly derivable from the known art, with the evident and required adaptations for the specific applications. Therefore, a detailed description thereof will be omitted, such an embodiment being within the capacity of a person skilled in the art.

The endoscope 10 comprises a central body where there is located a vision device 22, e.g. a camera, preferably a digital camera, for taking images related to the internal area of an organ.

It comprises a first working arm 21 for the use of tools, e.g. pincers, lancets, etc.

According to the present invention, the endoscope 10 further comprises a second working arm 23 for the use of tools in order to work on the examined organ.

The two working arms 21, 23 are both flexible and have a structure substantially analogous to that of the main body, hence having each a respective metal skeleton covered with a flexible rubber sheath.

Advantageously, the two working arms may be handled independently the one from the other.

Second handling means are provided in order to allow predetermined motions to the working arms 21, 23.

As is the case for the main body, such second handling means comprises a system of one or more tie rods, mechanically operated by a user.

Alternatively, or even in association to the tie rod system, such second handling means may advantageously comprise second motion actuating and controlling devices of electronic and/or electromechanical type.

In that case the motions of the two arms will be servoassisted and the user will control such motions via electronic controls, e.g. a pair of joystick 16 or the like.

In particular, each of the two working arms may slide longitudinally with respect to the main body, independently the one from the other, as indicated in FIG. 4 by the arrow F1 for the arm 21, and by the arrow F2 for the arm 23, respectively.

Moreover, the two arms can move transversally with respect to the main body, moving away and/or nearing the one with respect to the other, as it is indicated by arrow F3 of FIG. 5.

The endoscope further comprises a central body 25, the image-taking camera being connected at the working end thereof.

According to the preferred embodiment, the two working arms 21, 23 are located on two opposite sides of said central body and are connected thereto by one or more annular mechanisms 30, each one being apt to rotate about the central body, when suitably controlled by the operator, as it is illustrated in FIG. 6.

Each such annular mechanisms comprises a pair of slides in order to allow the two working arms to slide longitudinally with respect to the central body.

Such annular mechanisms are made so as to have a certain elasticity allowing also the transversal motions for the moving away/nearing of the two working arms with respect to the central body. This elasticity is attained e.g. through an elastically connected mesh structure.

Hence, such mechanisms allows the two working arms to move independently, so as to make them assume complex working configurations, as it is shown e.g. in FIGS. 7 and 8.

The vision device 22 housed in the central body 25 of the endoscope comprises one or more lenses, each thereof being apt to be actuated so as to vary its tilt with respect to the vision device.

According to the preferred embodiment of the present invention, such lenses are two and are tiltable.

Their arrangement onto the central body reunites the features hereto found separately on known endoscopes, i.e. the first lens has a front location whereas the second has a sideways location.

Their tilt, with respect to the vision device, is managed by means for adjusting the position of the lenses, of mechanic type, e.g. comprising a tie rod system operable by the operator, or of electromechanical and/or electronic type, with the aid of magnetostrictive sensors and actuators.

Moreover, the instrument according to the present invention comprises means 40 for processing and visualizing the images taken.

Such means advantageously allows the transfer of the images from the cameras to a computer 40, equipped with specific image reproduction software.

Such processing means are apt to provide stereoscopic images of the area taken and to visualize them, e.g., on a monitor.

This provides the operator with an extremely realistic view of the explored area, as if it were directly observed.

Thus, the operator may use the endoscope in search of the optimum position for operating inside of the organ explored, and not for finding the correct visual angle, as it often happens at present.

Upon finding such optimum position, the combined use of the two independent working arms allows the operator to carry out a large number of surgical interventions.

In fact, the availability of two flexible working arms, together with the central vision device, allows to operate simulating a manual surgical practice, recreating those angles between the two working arms otherwise not viable with common endoscopes equipped with a single working arm.

Moreover, an endoscope according to the present invention may be equipped with means for monitoring its position with respect to the inside of the explored organ.

Such monitoring means comprises one or more signal transmitters positioned on the main body and one or more external receivers of said signals, suitably externally positioned onto the patient's body.

The transmitters could, e.g., be transponders or other devices. The type of receiver will of course depend on the choice of design.

It is understood that such choices do not modify the operation principle of the present invention and should be construed as design choices within the capacity of a person skilled in the art, effected according to the specific embodiment.

The signals received by the receivers provide information on the instrument position inside of the organ.

Such information are advantageously used in order to graphically represent on a monitor the position of the instrument with respect to the explored organ, as it is shown by way of example in FIG. 9, so as to provide the operator with a real-time checking of the work done.

Moreover, the instrument may also be equipped with means for controlling the frictions between the instrument and the inside walls of the organ.

In particular, such controlling means comprises one or more pressure and/or force sensors, e.g. of membrane and/or of piezoelectric type.

The signals and the information obtained from said sensors are advantageously used to graphically represent the measured parameters on a monitor, providing indications facilitating and improving the operator's work.

The present invention was hereto described according to a preferred embodiment thereof, given by way of example and not for limitative purposes.

It is understood that other embodiments may be provided, all to be construed as falling within the protective scope thereof, as set forth by the annexed claims.

1-34. (canceled)

35. An endoscopic instrument having a flexible and elongate main body that houses a vision device for taking images of an organ internal area, comprising a first working arm for the use of tools and a second working arm for the use of tools apt to be operated independently with respect to said first working arm, characterized in that said first and second working arm are transversally movable with respect to said main body, moving away and/or nearing the one with respect to the other.

36. The instrument according to claim 35, wherein said main body comprises an annular metal skeleton coated with a rubber material sheath.

37. The instrument according to claim 35, further comprising first handling means of said main body.

38. The instrument according to claim 37, wherein said first handling means comprises one or more tie rods mechanically operated by a user.

39. The instrument according to claim 37, wherein said first handling means comprises first motion actuating and controlling devices of electronic and/or electromechanical type.

40. The instrument according to claim 35, wherein each of said first and second working arm are flexible.

41. The instrument according to claim 35, wherein each of said first and second working arm comprises a respective annular metal skeleton coated with a rubber material sheath.

42. The instrument according to claim 35, wherein each of said first and second working arm is apt to slide longitudinally with respect to said main body, independently the one from the other.

43. The instrument according to claim 35, further comprising second handling means of said first and second working arm.

44. The instrument according to claim 43, wherein said second handling means comprises one or more tie rods mechanically operated by a user.

45. The instrument according to claim 43, wherein said second handling means comprises second motion actuating and controlling devices of electronic and/or electromechanical type.

46. The instrument according to claim 35, wherein said main body comprises a central body, said vision device being connected at the end thereof.

47. The instrument according to claim 46, wherein said first and second working arm are located on two opposite sides of said central body.

48. The instrument according to claim 46, wherein said first and second working arm are connected to said central body by one or more annular mechanisms, each of said mechanisms being apt to rotate about said central body.

49. The instrument according to claim 48, wherein each of said annular mechanisms is made with an elastically connected mesh structure.

50. The instrument according to claim 35, wherein said vision device comprises a camera.

51. The instrument according to claim 50, wherein said camera is of digital type.

52. The instrument according to claim 35, wherein said vision device comprises one or more lenses.

53. The instrument according to claim 52, wherein each of said lenses is apt to be handled so as to vary its tilt with respect to the vision device.

54. The instrument according to claim 53, comprising two tiltable lenses.

55. The instrument according to claim 54, comprising means for adjusting the position of said lenses.

56. The instrument according to claim 55, wherein said adjusting means are of mechanic type, comprising a tie rod system operable by a user.

57. The instrument according to claim 55, wherein said adjusting means are of electromechanical and/or electronic type.

58. The instrument according to claim 35, further comprising means for processing and visualizing the images taken.

59. The instrument according to claim 58, wherein said processing and visualizing means are apt to provide stereoscopic images of the area taken.

60. The instrument according to claim 35, further comprising means for monitoring its position with respect to said organ.

61. The instrument according to claim 60, wherein said monitoring means comprises one or more signal transmitters positioned on said main body and one or more external receivers of said signals, said received signals being representative of the position of the instrument.

62. The instrument according to claim 61, wherein said transmitters comprise one or more magnetic field coils.

63. The instrument according to claim 61, wherein said transmitters comprise one or more transponders.

64. The instrument according to claim 35, further comprising means for controlling frictions between the instrument and said organ.

65. The instrument according to claim 64, wherein said means for controlling frictions comprises one or more pressure and/or force sensors.

66. The instrument according to claim 65, wherein one or more of said sensors is of piezoelectric type.

67. The instrument according to claim 65, wherein one or more of said sensors is of membrane type.

68. The instrument according to claim 64, further comprising means for graphically representing said position of the instrument with respect to the organ and said frictions.
